(19) United States
(12) Patent Application Publication
(10) Pub. No.: US 2008/0176793 A1
(43) Pub. Date: Jul. 24, 2008

(54) UPREGULATING ACTIVITY OR EXPRESSION OF BDNF TO MITIGATE COGNITIVE IMPAIRMENT IN ASYMPTOMATIC HUNTINGTON'S SUBJECTS

(75) Inventors: Danielle Simons, Laguna Beach, CA (US); Gary Lynch, Irvine, CA (US); Eniko Kramar, Irvine, CA (US)

Correspondence Address: BEYER WEAVER LLP, P.O. BOX 70250, OAKLAND, CA, 94612-0250, US

(73) Assignee: THE REGENTS OF THE UNIVERSITY OF CALIFORNIA, Oakland, CA (US)

(21) Appl. No.: 11/857,190

(22) Filed: Sep. 18, 2007

Related U.S. Application Data
(60) Provisional application No. 60/845,611, filed on Sep. 18, 2006.

(51) Int. Cl. A61K 38/35; A61K 31/137; A61K 31/55; A61K 31/435; A61K 31/22; A61K 38/02; A61K 31/4439; A61K 31/405; A61K 31/197; A61K 31/554; A61K 31/138; A61K 31/439; A61K 31/404; A61K 31/565

(52) U.S. Cl. 514/12; 514/651; 514/217; 514/277; 514/546; 514/2; 514/343; 514/211.13; 514/648; 514/295; 514/414; 514/182; 514/643; 514/567; 514/415

(57) ABSTRACT

This invention provides novel methods of treatment to ameliorate or prevent cognitive disorder/dysfunction in pre- or asymptomatic subject having one or more mutations in the Huntington gene. The methods involve increasing the expression or activity of the neurotrophin BDNS in the brain of said subject.

### CROSS-REFERENCE TO RELATED APPLICATIONS

This application claims benefit of and priority to U.S. Ser. No. 60/845,611, filed on Sep. 18, 2006, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was funded, in part, by US National Institutes of Health grants NS051823 and NS045260. The Government of the United States of America has certain rights in this invention.

### FIELD OF THE INVENTION

This invention pertains to the field of Huntington's disease and associated cognitive disorders. In particular this invention pertains to the treatment of pre- or asymptomatic Huntington's subjects to reduce or prevent cognitive dysfunction associated with later disease progression.

### BACKGROUND OF THE INVENTION

The clinical management of numerous neurological disorders has been frustrated by the progressive nature of degenerative, traumatic, or destructive neurological diseases and the limited efficacy and serious side-effects of available pharmacological agents. Conditions such as Huntington's disease, Alzheimer's disease, severe seizure disorders (e.g., epilepsy and familial dysautonomia), as well as injury or trauma to the nervous system have eluded most conventional pharmacological attempts to alleviate or cure the conditions.

Huntington's disease has proven particularly elusive to conventional pharmacological treatments. Huntington's disease is a progressive degenerative disease of the basal ganglia that is inherited as an autosomal dominant trait. The onset of Huntington's disease, an autosomal dominant, neurodegenerative disorder occurs at an average age of 35 to 40 years but can occur in persons as young as two years old or as old as 80 years.

The onset is insidious and is characterized by abnormalities of coordination, movement, and behavior. Movement abnormalities include restlessness, mild postural abnormalities, and quick jerking movements of the fingers, limbs, and trunk. The movement abnormalities may be accompanied by substantial weight loss. Depression is common, and cognitive abnormalities and inappropriate behavior may develop. In contrast to the choreic movements typical of onset in adults, juvenile patients may exhibit rigidity, tremor, and dystonia. In the course of eight to 15 years, the disorder progresses to complete incapacitation, with most patients dying of aspiration pneumonia or inanition.

Huntington's disease was the first major inherited disorder with an unidentified basic defect to be linked with a DNA marker (Gusella et al. (1983) Nature 306: 234). The product of this gene, designated huntingtin, contains more than 3000 amino acids and is encoded by 10,366 bases at 4p16.3 (Huntington's Disease Collaborative Research Group (1993) Cell 72: 971). Although knowledge of the underlying molecular basis for Huntington's disease has increased in recent years, pharmacological treatments based on this molecular knowledge have been limited to alleviating some of the symptoms associated with HD, a procedure that does not address the primary degenerative process nor the nonmotor aspects of the disease.

### SUMMARY OF THE INVENTION

This invention pertains to the discovery that long-term potentiation (LTP), regarded as a substrate for memory encoding, is severely impaired early in the disease progression in presymptomatic Huntington's Disease (HD) mutant mice and by implication in presymptomatic Huntington's Disease humans. Importantly, these HD-specific deficits in LTP are reversed by delivering the neurotrophin, Brain-Derived Neurotrophic Factor (BDNF). BDNF is reduced in HD patients and mutant mice and is a potent facilitator of LTP. Thus, for the first time, these discoveries show that increasing BDNF levels and/or triggering endogenous receptors that stimulate and/or mimic the actions of BDNF will ameliorate the cognitive deficits associated with HD. Furthermore, cognitive deficits, especially those involving memory, are present in asymptomatic HD gene carriers thus up-regulating BDNF in these patients represents a novel indication for treatment.

Accordingly, in certain embodiments, this invention provides a method of preserving or improving cognitive function in a presymtomatic or asymptomatic mammal having one or more mutations predisposing the mammal to Huntington's disease. The method typically comprises maintaining or increasing the BDNF level or activity in the brain of the mammal. In certain embodiments the mammal is a mammal that shows no substantial neural degeneration. In certain embodiments the mammal shows essentially no measurable neural degeneration. In certain embodiments the mammal is a mammal diagnosed as having one or more mutations in the huntingtin gene. In certain embodiments the mammal is a mammal diagnosed as having one or more mutations in the huntingtin gene prior to maintaining or increasing the BDNF level or activity. In certain embodiments the mammal is a mammal not having a diagnosis and/or treatment for depression. In certain embodiments the mammal is a mammal not having a diagnosis of depression and/or other psychiatric disorder. In certain embodiments the mammal is a human (e.g. a human adult, a human adolescent, a human child, etc.) diagnosed as having one or more mutations in the huntingtin gene prior to maintaining or increasing the BDNF level or activity. In certain embodiments the mutation is a trinucleotide repeat expansion in the huntingtin gene. In certain embodiments the maintaining or increasing the BDNF level or activity comprises administering a glutamate AMPA receptor modulators (ampakines) to the mammal in an amount sufficient to upregulate expression or activity of BDNF in the mammal. In certain embodiments maintaining or increasing the BDNF level or activity in the mammal comprises restricting diet and/or increasing physical exercise of the mammal. In certain embodiments maintaining or increasing the BDNF level or activity in the mammal comprises administering to the mammal one or more agents selected from the group consisting of an anti-depressant drug or an anti-anxiolytic drug, an anti-psychotic drug, an acetylcholinesterase inhibitor. In certain embodiments the agent comprises fluoxetine, desipramine, or 2-methyl-6-(phenylethynyl)-pyridine). In certain embodiments the agent comprises afobazole. In certain embodiments the agent comprises a histone deacetylase inhibitors (e.g. sodium butyrate). In certain embodiments the agent comprises a neuropeptide whose expression is regulated by cocaine- or other amphetamine. In certain embodiments the agent comprises cystamine or nicotine (but the treatment is not smoking or tobacco use). In certain embodiments the agent comprises quetiapene or venlafaxine. In certain embodiments the agent comprises huperzine A. In certain embodiments the agent comprises a monocyclic or bicyclic loop mimetic of BDNF. In certain embodiments the agent comprises estrogen or adrenocorticotropin. In certain embodiments the agent comprises dopamine, norepinephrine, LDOPA, serotonin, or analogues thereof. In certain embodiments the agent comprises Semax. In certain embodiments the agent comprises a compound that increases the activity of BDNF through up-regulating the BDNF receptor.

Also provided is the use of a compound that increases the level or activity of BDNF in a mammal in the manufacture of a medicament for the treatment or prevention of cognitive dysfunction in a pre- or asymptomatic mammal having one or more mutations in the Huntington gene. In certain embodiments, the compound is an ampakine.

Also provided is a kit for the treatment or prevention of cognitive dysfunction in a pre- or asymptomatic mammal having one or more mutations in the Huntington gene, the kit comprising: a container containing one or more agents that increase the expression or activity of BDNF in a mammal (e.g., ampakines); and instructional materials teaching the use of the agents to mitigate or prevent cognitive disorder in a presymptomatic or asymptomatic mammal diagnosed with one or more mutations in a Huntington gene.

### DEFINITIONS

The term “cyano” refers to the group —CN.

The terms “Halogen” or “halo” refer to fluorine, bromine, chlorine, and iodine atoms.

The terms “thiol” or “mercapto” refers to the group —SH.

The term “sulfamoyl” refers to the —SO₂NH₂.

The term “alkyl” refers to a cyclic, branched or straight chain, alkyl group of one to eight carbon atoms. The term “alkyl” includes reference to both substituted and unsubstituted alkyl groups. This term is further exemplified by such groups as methyl, ethyl, n-propyl, i-propyl, n-butyl, t-butyl, i-butyl (or 2-methylpropyl), cyclopropylmethyl, cyclohexyl, i-amyl, n-amyl, and hexyl. Substituted alkyl refers to alkyl as just described including one or more functional groups such as aryl, acyl, halogen, hydroxyl, amido, amino, acylamino, acyloxy, alkoxy, cyano, nitro, thioalkyl, mercapto and the like. These groups may be attached to any carbon atom of the lower alkyl moiety. “Lower alkyl” refers to C₁-C₆ alkyl, with C₁-C₄ alkyl more preferred. “yclic alkyl” includes both mono-cyclic alkyls, such as cyclohexyl, and bi-cyclic alkyls, such as [3.3.0]bicyclooctane and [2.2.1]bicycloheptane. “Fluoroalkyl” refers to alkyl as just described, wherein some or all of the hydrogens have been replaced with fluorine (e.g., —CF₃ or —CF₂CF₃).

The terms “aryl” or “Ar” refers to an aromatic substituent which may be a single ring or multiple rings which are fused together, linked covalently, or linked to a common group such as an ethylene or methylene moiety. The aromatic ring(s) may contain a heteroatom, such as phenyl, naphthyl, biphenyl, diphenylmethyl, 2,2-diphenyl-1-ethyl, thienyl, pyridyl and quinoxalyl. The term “aryl” or “Ar” includes reference to both substituted and unsubstituted aryl groups. If substituted, the aryl group may be substituted with halogen atoms, or other groups such as hydroxy, cyano, nitro, carboxyl, alkoxy, phenoxy, fluoroalkyl and the like. Additionally, the aryl group may be attached to other moieties at any position on the aryl radical which would otherwise be occupied by a hydrogen atom (such as 2-pyridyl, 3-pyridyl and 4-pyridyl).

The term “alkoxy” denotes the group .quadrature.OR, where R is lower alkyl, substituted lower alkyl, aryl, substituted aryl, aralkyl or substituted aralkyl as defined below.

The term “acyl” denotes groups —C(O)R, where R is alkyl, substituted alkyl, alkoxy, aryl, substituted aryl, amino and alkylthiol.

A “carbocyclic moiety” denotes a ring structure in which all ring vertices are carbon atoms. The term encompasses both single ring structures and fused ring structures. Examples of aromatic carbocyclic moieties are phenyl and naphthyl.

The term “heterocyclic moiety” denotes a ring structure in which one or more ring vertices are atoms other than carbon atoms, the remainder being carbon atoms. Examples of non-carbon atoms are N, O, and S. The term encompasses both single ring structures and fused ring structures. Examples of aromatic heterocyclic moieties are pyridyl, pyrazinyl, pyrimidinyl, quinazolyl, isoquinazolyl, benzofuryl, isobenzofuryl, benzothiofuryl, indolyl, and indolizinyl.

The term “amino” denotes the group NRR′, where R and R′ may independently be hydrogen, lower alkyl, substituted lower alkyl, aryl, substituted aryl as defined below or acyl.

The term “amido” denotes the group —C(O)NRR′, where R and R′ may independently be hydrogen, lower alkyl, substituted lower alkyl, aryl, substituted aryl as defined below or acyl.

The term “independently selected” is used herein to indicate that the two R groups, R¹ and R², may be identical or different (e.g., both R¹ and R² may be halogen or, R¹ may be halogen and R² may be hydrogen, etc.).

The term “subject” means a mammal, particularly a human. The term specifically includes domestic and common laboratory mammals, such as non-human primates, felines, canines, equines, porcines, bovines, goats, sheep, rabbits, rats and mice.

“Alpha-amino-3-hydroxy-5-methyl-isoxazole-4-propionic acid”, or “AMPA”, or “glutamatergic” receptors are molecules or complexes of molecules present in cells, particularly neurons, usually at their surface membrane, that recognize and bind to glutamate or AMPA. The binding of AMPA or glutamate to an AMPA receptor normally gives rise to a series of molecular events or reactions that result in a biological response. The biological response may be the activation or potentiation of a nervous impulse, changes in cellular secretion or metabolism, or causing cells to undergo differentiation or movement.

The phrase “effective amount” means a dosage sufficient to produce a desired result.

### BRIEF DESCRIPTION OF THE DRAWINGS

FIG. 1 shows that long-term potentiation is impaired in Hdh^{Q92} and Hdh^{Q111} mice. Theta burst stimulation caused an immediate increase in the slope of the fEPSPs in wild-type (WT) mice, after which the responses stabilized at a level about 40% above baseline. In both Hdh^{Q92} and Hdh^{Q111} mice (Hdh), the initial induction of LTP was reduced and responses decayed steadily back to baseline over 40 min. Means ±s.e.m are shown for this and subsequent graphs.

FIG. 2, panels a-d, show that Facilitation of burst responses during TBS is impaired in Hdh^{Q111} mice. Panel a: Shown are the responses to first and fourth stimulation bursts of a theta train from WT (top) and Hdh^{Q111} (bottom) slices. Note that for each genotype the first response (‘burst 1’) is similar and the fourth response (‘burst 4’) is larger than the first; however, between-burst facilitation is more pronounced in the WT slice. Panel b: Graph depicts the sizes of responses to stimulation bursts 2-10 in a theta train expressed as a fraction of the area of the composite response to the first burst. The large facilitation of bursts 2-10 seen in slices from WT mice is greatly attenuated those from Hdh^{Q111} mice (mean facilitation across bursts 2-10; P=0.03). Panel c: The IPSC refractory period is well developed in Hdh^{Q111} slices. Note that the second of two IPSCs induced by a pair of stimulation pulses (interpulse interval of 200 msec) is substantially reduced in Hdh^{Q111} mice, as it is in WTs (not shown). Panel d: Paired stimulation pulses delivered with the indicated inter-pulse intervals showed that the IPSC refractory effect is time-dependent and does not differ between WT and Hdh^{Q111} slices (size of the second IPSC is plotted as fraction of the first)

FIG. 3, panels a-d, illustrate TBS-induced actin polymerization in dendritic spines. Survey photomicrographs showing CA1b stratum radiatum of WT hippocampal slices that received either low frequency stimulation or TBS to the Schaffer-commissural fibers. The pyramidal cell layer is at the top of the photomicrographs. Panel a: Low frequency stimulation generated very few structures with intense rhodamine-phalloidin labeling. Panel b: LTP, induced by TBS, was accompanied by the presence of numerous intensely labeled puncta. Panel c: At higher magnification, clusters of TBS-induced, phalloidin-labeled puncta are evident along the dendrite segment. Some puncta are connected to the dendrite by a lightly labeled, thin neck. Panel d: High magnification photomicrograph from a second slice given TBS. Scale bars: 10 μm.

FIG. 4, panels a-d, show that polymerized actin co-localizes with synaptic markers. Photomicrographs show in situ phalloidin labeling and PSD-95 immunostaining in CA1b stratum radiatum of WT mice. Panel a: Survey micrograph showing phalloidin labeling (red) and PSD-95 immunostaining (green) following LTP induction in a WT slice. The arrow and arrowhead point to the same sites shown at higher magnification in subsequent panels. (Panels b and c) High magnification photomicrographs show PSD-95 immunostaining (panel b) and phalloidin labeling (panel c) in the field shown in panel (a). Panel d: Overlay of panels (b) and (c) showing that phalloidin and PSD-95 are co-localized in some puncta (yellow structures; arrow); other phalloidin-labeled puncta are ‘capped’ by PSD-95 immunostaining (arrowhead). Scale bars: 10 μm in panel a; 3 μm in panels b-d.

FIG. 5, panels a-c, show that actin polymerization in dendritic spines is greatly reduced in Hdh^{Q111} mice. Phalloidin-labeling of CA1 stratum radiatum of hippocampal slices from WT and Hdh^{Q111} mice after TBS-induced LTP. Panel a: Densely-labeled spines are abundant in WT slices following TBS. The pyramidal cell layer is at the top left of the photomicrograph. Panel b: Comparable photomicrograph from a Hdh^{Q111} slice. Panel c: The maximum number of spines in three low intensity bins compared to those in three high intensity bins in WT and Hdh^{Q111} (Hdh) slices receiving low frequency stimulation (LFS) or TBS. TBS generated a marked increase in the number of densely labeled spines in WT but not Hdh^{Q111} slices. Note that the low intensity values are comparable for WT and Hdh slices receiving LFS or TBS. *P=0.009 compared to WT, LFS; #p=0.02 compared to WT, TBS. Scale bars: 10 μm.

FIG. 6, panels a and b, show that production of BDNF protein is reduced in hippocampus of eight week-old Hdh^{Q111} mice. Panel a: Representative western blot prepared from hippocampal homogenates shows that both pro-BDNF (pBDNF) and mature BDNF (mBDNF) levels are reduced in Hdh^{Q111} mice relative to WTs. Samples for two mice from each genotype are shown. The farthest left lane shows the migration of recombinant BDNF (rBDNF) protein. The actin immunoband from the stripped and re-probed blot is shown at the bottom. Panel b: Densitometric analyses confirmed that both pBDNF and mBDNF are reduced in Hdh^{Q111} relative to WT hippocampus (**P=0.0001 and *P=0.002, respectively). The decrease in pBDNF in the Hdh^{Q111} mice was larger than that for mBDNF (#P=0.007).

FIG. 7, panels a-d, show that BDNF restores TBS-induced LTP and actin polymerization, but not burst response facilitation in Hdh^{Q111} mice. Panel a: LTP in untreated Hdh^{Q111} slices (Hdh) did not stabilize. However, in the presence of BDNF (2 nM), LTP in slices from the same animals (Hdh+BDNF) was not detectably different from WTs. Panel b: fEPSPs in a subgroup of BDNF-treated Hdh^{Q111} slices were recorded for 60 min after TBS. Note that potentiation was still present at the end of recording (data for the untreated Hdh^{Q111} slices from FIG. 1 are shown for comparison). Panel c: BDNF, at concentrations that rescued LTP, did not correct the impaired theta burst facilitation seen in Hdh^{Q111} mice, as evidenced by comparable burst facilitation in BDNF-treated and untreated Hdh^{Q111} slices (sizes of burst responses 2-10 are expressed as a fraction of the burst response 1). Panel d: Phalloidin labeling in CA1 stratum radiatum of BDNF-treated and untreated Hdh^{Q111} slices. Densely labeled spines were largely absent in untreated slices after TBS (top panel) but were present in large numbers in BDNF-treated cases (bottom panel). Scale bar: 10 μm.

FIG. 8 illustrates compounds in accordance with Formula I of U.S. Pat. No. 6,166,008.

FIG. 9 illustrates compounds in accordance with Formula II of U.S. Pat. No. 6,166,008.

FIG. 10 illustrates compounds in accordance with Formula III of U.S. Pat. No. 6,166,008.

FIG. 11 shows the structure of compound CX516, 1-(Quinoxalin-6-ylcarbonyl)piperidine,

### DETAILED DESCRIPTION

This invention pertains to the discovery that long-term potentiation (LTP), regarded as a substrate for memory encoding, is severely impaired early in the disease progression in presymptomatic Huntington's Disease (HD) mutant mice and by implication in presymptomatic Huntington's Disease humans. Importantly, these HD-specific deficits in LTP are reversed by delivering the neurotrophin, Brain-Derived Neurotrophic Factor (BDNF). BDNF is reduced in HD patients and mutant mice and is a potent facilitator of LTP. Thus, for the first time, these discoveries show that increasing BDNF levels and/or triggering endogenous receptors that stimulate and/or mimic the actions of BDNF will ameliorate the cognitive deficits associated with HD. Furthermore, cognitive deficits, especially those involving memory, are present in asymptomatic HD gene carriers thus up-regulating BDNF in these patients represents a novel indication for treatment.

This invention thus provides therapeutic strategies for improving cognitive, and cognition-related, deficits associated with Huntington's Disease (HD). These deficits occur in asymptomatic gene carriers of HD before the motor symptoms, that characterize the disease manifest. To our knowledge, this is the first suggestion that pre-symptomatic HD patients can be treated. The therapeutic/prophylactic strategies involve increasing levels and/or activity of the neurotrophin, Brain-Derived Neurotrophic Factor (BDNF), and thereby modulating properties associated with long-term potentiation (LTP), which is regarded as a substrate for memory encoding. This provides methods of treating, preventing, and/or alleviating the cognitive deficits in HD, some of which occur very early in the progression of the disease.

In particular, the prophylactic/therapeutic methods of this invention typically involve increasing BDNF levels and/or activity. Without being bound to a particular theory it is believed this provides a novel strategy for improving deficits in learning and memory as well as other higher order behaviors in asymptomatic gene carriers. To date no treatments and/or therapeutics methods for these HD-associated cognitive conditions exist.

Moreover, this invention provides a mechanism-based strategy for the treatment of HD as the instant invention shows that LTP deficits in the hippocampus (a brain area involved in learning and memory) occur very early in the disease progression of HD. These deficits parallel decreases in the BDNF protein.

Importantly, it is shown herein (see, e.g., Example 1) that delivering BDNF to the hippocampus of presymptomatic HD mutant mice during ex vivo electrophysiological recording restores LTP. Since various strategies, methods, and compounds exist for increasing BDNF levels and/or activity, increasing BDNF and/or related neurotrophins is a feasible therapeutic manipulation for treating cognitive, and cognition-related, deficits associated with HD.

Various methods of increasing BDNF levels include, but are not limited to glutamate AMPA receptor modulators (e.g. ampakines) (see, e.g., U.S. Pat. No. 6,030,968 and US 2005/0228019 A1, which are incorporated herein by reference, e.g., for the compounds disclosed therein), physical exercise, dietary restriction, anti-depressant drugs (e.g. fluoxetine, desipramine, 2-methyl-6-(phenylethynyl)-pyridine), anti-anxiolytics (e.g. afobazole), histone deacetylase inhibitors (e.g. sodium butyrate), neuropeptides (e.g. cocaine- and amphetamine-regulated transcript), cystamine and related agents, nicotine, anti-psychotics (e.g. quetiapene, venlafaxine), and acetylcholinesterase inhibitors (e.g. huperzine A).

In certain embodiments a wide variety of AMPA receptor potentiators are useful in the present invention, including ampakines (disclosed in International Patent Application Publication No. WO 94/02475 (PCT/US93/06916), U.S. Pat. Nos. 5,773,434, 6,274,600, and 6,166,008, which are herein incorporated by reference in their entirety for all purposes; LY404187, LY 392098, LY503430, and derivatives thereof (produced by Eli Lilly, Inc.); CX546 and derivatives thereof; CX614 and derivatives thereof; S18986-1 and derivatives thereof; benzoxazine AMPA receptor potentiators and derivatives thereof (as disclosed in U.S. Pat. Nos. 5,736,543, 5,962,447, 5,773,434 and 5,985,871 which are herein incorporated by reference in their entirety for all purposes); heteroatom substituted benzoyl AMPA receptor potentiators and derivatives thereof (e.g., as disclosed in U.S. Pat. Nos. 5,891,876, 5,747,492, and 5,852,008, which are herein incorporated by reference in their entirety for all purposes); benzoyl piperidines/pyrrolidines AMPA receptor potentiators and derivatives thereof as (e.g. as disclosed in U.S. Pat. No. 5,650,409, which is herein incorporated by reference in its entirety for all purposes); benzofurazan carboxamide AMPA receptor potentiators and derivatives thereof (e.g., as disclosed in U.S. Pat. Nos. 6,110,935, 6,313,1315 and 6,730,677 which are incorporated herein by reference); 7-chloro-3-methyl-3-4-dihydro-2H-1,2,4 benzothiadiazine S,S, dioxide and derivatives thereof (e.g., as described by Zivkovic et al. (1995) J. Pharmacol. Exp. Therap., 272: 300-309; and Thompson et al. (1995) Proc. Natl. Acad. Sci., USA, 92:7667-7671).

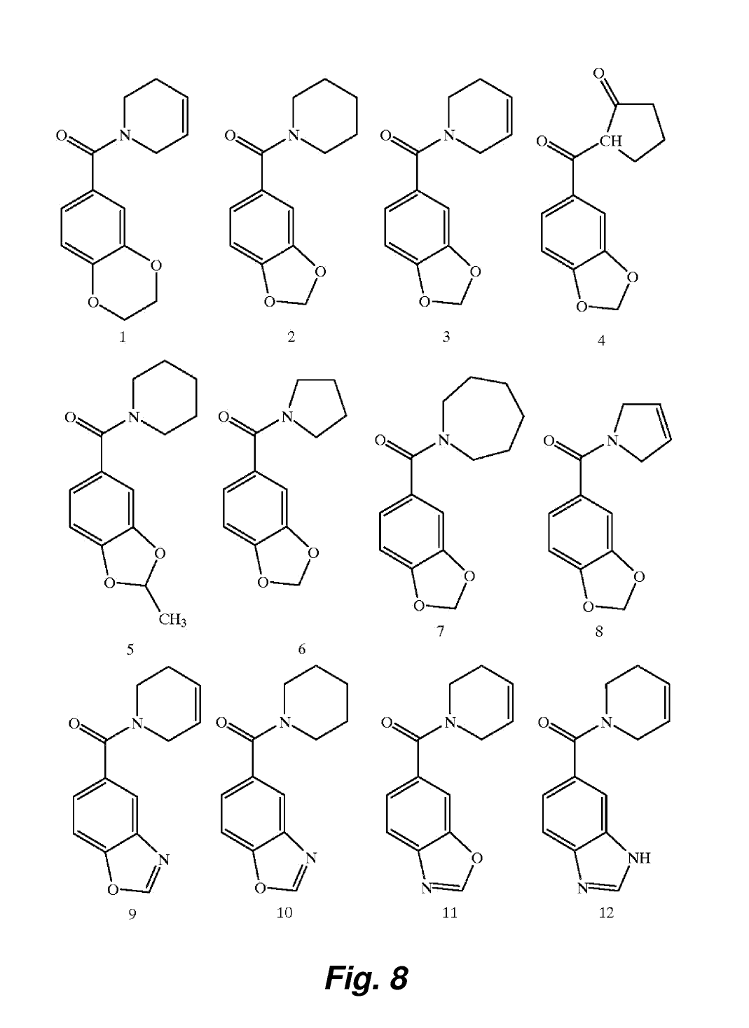

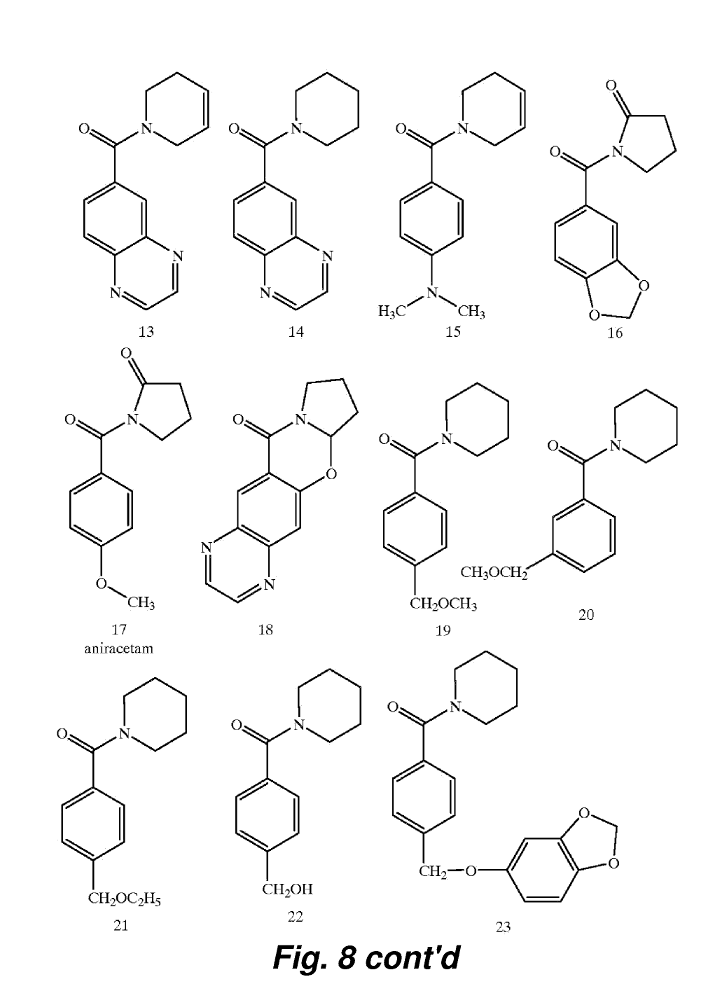

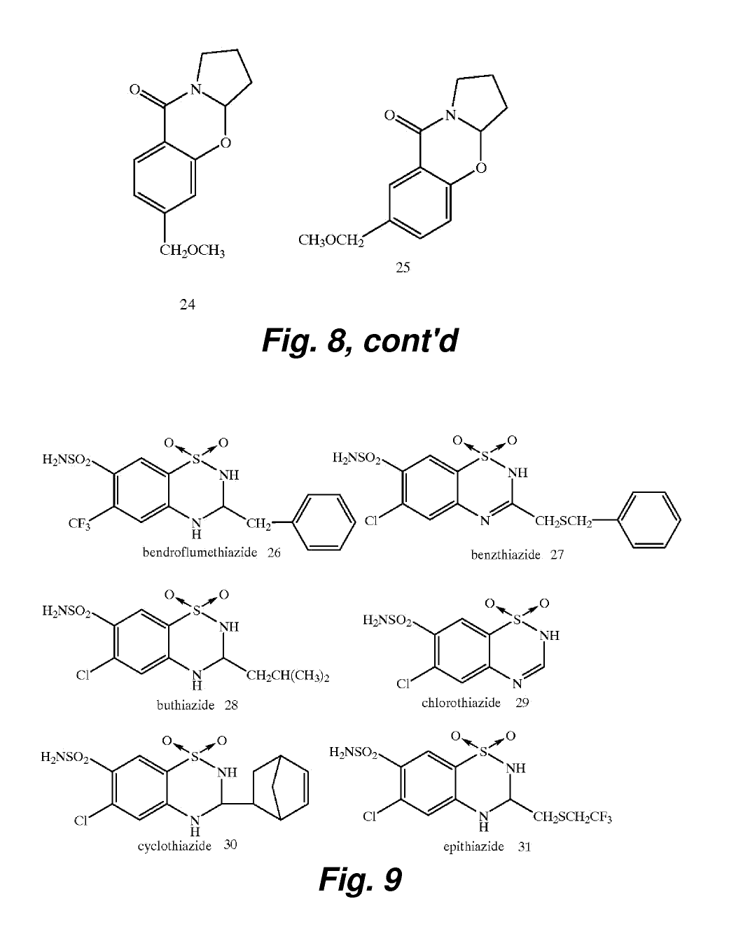

In certain embodiments the methods of this invention utilize ampakines as described, for example, in U.S. Pat. No. 6,166,008. Such ampakines include, ampakines according to formula I of U.S. Pat. No. 6,166,008:

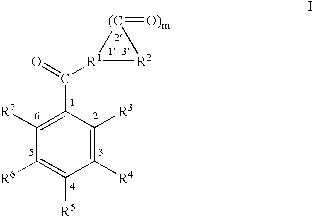

in which: R¹ is a member selected from the group consisting of N and CH; m is O or 1; R² is a member selected from the group consisting of (CR⁸₂)ₙ₋ₘ and Cₙ₋ₘR⁸₂₍ₙ₋ₘ₎₋₂, in which n is 4, 5, 6, or 7, the R⁸'s in any single compound being the same or different, each R⁸ being a member selected from the group consisting of H and C₁-C₆ alkyl, or one R⁸ being combined with either R³ or R⁷ to form a single bond linking the no. 3′ ring vertex to either the no. 2 or the no. 6 ring vertices or a single divalent linking moiety linking the no. 3′ ring vertex to either the no. 2 or the no. 6 ring vertices, the linking moiety being a member selected from the group consisting of CH₂, CH₂—CH₂, CH═CH, O, NH, N(C₁-C₆ alkyl), N═CH, N═C(C₁-C₆ alkyl), C(O), O—C(O), C(O)—O, CH(OH), NH—C(O), and N(C₁-C₆ alkyl)-C(O); R³, when not combined with any R⁸, is a member selected from the group consisting of H, C₁-C₆ alkyl, and C₁-C₆ alkoxy; R⁴ is either combined with R⁵ or is a member selected from the group consisting of H, OH, and C₁-C₆ alkoxy; R⁵ is either combined with R⁴ or is a member selected from the group consisting of H, OH, C₁-C₆ alkoxy, amino, mono(C₁-C₆ alkyl)amino, di(C₁-C₆ alkyl)amino, and CH₂ OR⁹, in which R⁹ is a member selected from the group consisting of H, C₁-C₆ alkyl, an aromatic carbocyclic moiety, an aromatic heterocyclic moiety, an aromatic carbocyclic alkyl moiety, an aromatic heterocyclic alkyl moiety, and any such moiety substituted with one or more members selected from the group consisting of C C₁-C₃ alkyl, C₁-C₃ alkoxy, hydroxy, halo, amino, alkylamino, dialkylamino, and methylenedioxy; R⁶ is either H or CH₂ OR⁹; R⁴ and R⁵, when combined, form a member selected from the group consisting of

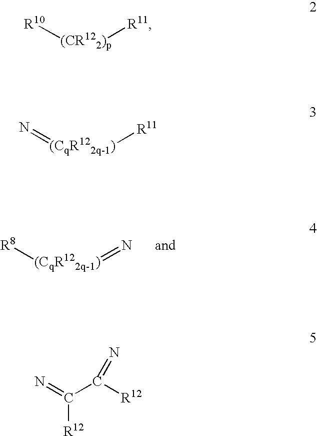

in which: R¹⁰ is a member selected from the group consisting of O, NH and N(C₁-C₆ alkyl); R¹¹ is a member selected from the group consisting of O, NH and N(C₁-C₆ alkyl); R¹² is a member selected from the group consisting of H and C₁-C₆ alkyl, and when two or more R¹²'s are present in a single compound, such R¹²'s are the same or different; p is 1, 2, or 3; and q is 1 or 2; and R⁷, when not combined with any R⁸, is a member selected from the group consisting of H, C₁-C₆ alkyl, and C₁-C₆ alkoxy. Compounds 1 through 25 in FIG. 8 are illustrative embodiments of compounds according to Formula I.

In another embodiment the ampakines are ampakines according to formula II of U.S. Pat. No. 6,166,008:

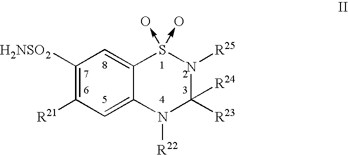

in which R²¹ is either H, halo or CF₃; R²² and R²³ either are both H or are combined to form a double bond bridging the 3 and 4 ring vertices; R²⁴ is either H, C₁-C₆ alkyl, C₅-C₇ cycloalkyl, C₅-C₇ cycloalkenyl, Ph (Ph denotes a phenyl group), CH₂Ph, CH₂SCH₂Ph, CH₂X, CHX₂, CH₂ SCH₂ CF₃, CH₂ SCH₂CH—CH₂, or

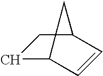

and R²⁵ is a member selected from the group consisting of H and C₁-C₆ alkyl.

Within the scope of Formula II, certain subclasses are preferred. One of these is the subclass in which R²¹ is C₁ or CF₃, with Cl preferred. Another is the subclass in which all X's are Cl. Still another is the subclass in which R²² and R²³ are both H. A preferred subclass of R²⁴ is that which includes CH₂Ph, CH₂SCH₂Ph, and

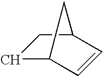

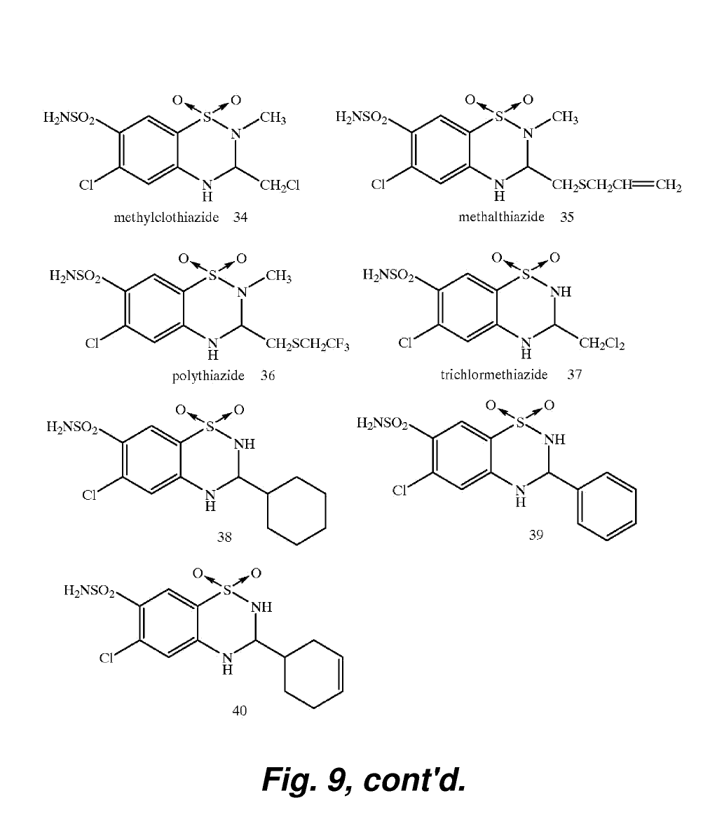

Compounds 26 through 40 in FIG. 9 are illustrative embodiments of compounds according to Formula II.

Certain preferred compounds within the scope of Formula II include those in which R²⁴ is either C₅-C₇ cycloalkyl, C₅-C₇ cycloalkenyl or Ph (“Ph” denotes a phenyl group). Other preferred compounds of this group are those in which R²¹ is halo, R²² is H, R²³ is H, and R²⁵ is H. Preferred substituents for R²⁴ include cyclohexyl, cyclohexenyl, and phenyl.

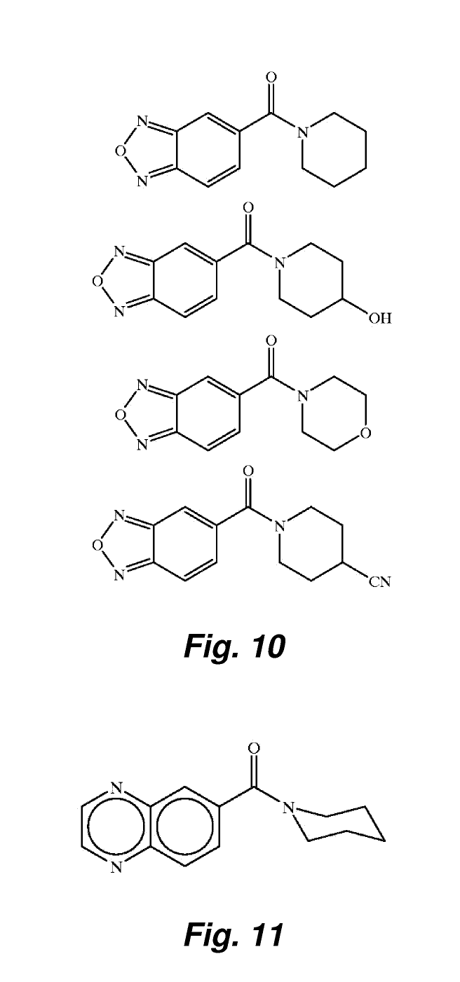

In another embodiment the ampakines are ampakines according to formula III of U.S. Pat. No. 6,166,008:

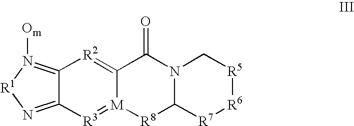

in which: R¹ is oxygen or sulfur; R² and R³ are independently selected from the group consisting of —N═, —CR═, and —CX═; M is ═N or ═CR⁴—, where R⁴ and R⁸ are independently R or together form a single linking moiety linking M to the ring vertex 2′, the linking moiety being selected from the group consisting of a single bond, —CR₂—, —CR═CR—, —C(O)—, —O—, —S(O)_{y}—, —NR—, and —N═; R⁵ and R⁷ are independently selected from the group consisting of —(C₂)ₙ—, —C(O)—, —CR═CR—, —CR═CX—, —C(RX)—, —CX₂—, —S—, and —O—; and R₆ is selected from the group consisting of —(CR₂)ₘ—, —C(O)—, —CR═CR—, —C(RX)—, —CR₂—, —S—, and —O—; where X is —Br, —Cl, —F, —CN, —NO₂, —OR, —SR, —NR₂, —C(O)R—, —CO₂R, or —CONR₂; and R is hydrogen, C₁-C₆ branched or unbranched alkyl, which may be unsubstituted or substituted with one or more functionalities defined above as X, or aryl, which may be unsubstituted or substituted with one or more functionalities defined above as X; m and p are independently 0 or 1; n and y are independently 0, 1 or 2. Certain preferred embodiments include, but are not limited to the compounds in FIG. 10.

One particularly preferred compound is compound CX516, 1-(Quinoxalin-6-ylcarbonyl)piperidine, having the structure shown in FIG. 11.

The compounds described above are prepared by conventional methods known to those skilled in the art of synthetic organic chemistry. Numerous synthetic methods are described in U.S. Pat. No. 6,166,008 and the references cited therein.

Other compounds for use in the methods of this invention include compounds that mimic the effects of BDNF. Such compounds include, but are not limited to peptides that are monocyclic and bicyclic loop mimetics of the neurotrophin. Furthermore, neurohormones (e.g. estrogen, adrenocorticotropin) and neurotransmitters and their precursors (e.g. dopamine, norepinephrine, LDOPA, serotonin) can up-regulate BDNF as well as compounds that mimic or increase levels of these neurochemicals (e.g. Semax is an analogue of the neurohormone adrenocorticotropin that increases BDNF levels). Finally, compounds that increase the activity of BDNF possibly through up-regulating its receptor (e.g. kinase inhibitors) are also viable therapeutics.

### Administration of Compounds

The various compounds described herein are administered in accordance with standard methods know to those of skill in the art. For example, the ampakines described herein can be incorporated into a variety of formulations for therapeutic administration. Examples include, but are not limited to are capsules, tablets, syrups, suppositories, and various injectable forms. Administration of the compounds is achieved in various ways, including oral, bucal, rectal, parenteral, intraperitoneal, intradermal, transdermal, nasal, etc., administration. In certain embodiments preferred formulations of the compounds are oral preparations, particularly capsules or tablets.

The above described compounds and/or compositions are administered at a dosage that preserves or improves cognitive function in a presymtomatic or asymptomatic mammal having or at risk for Huntington's disease (e.g., having one or more mutations predisposing said mammal to Huntington's disease), in presymtomatic or asymptomatic mammal, while at the same time minimizing any side-effects. It is contemplated that the composition will be obtained and used under the guidance of a physician.

Typical dosages for systemic Ampakine administration range from about 0.1 to about 1000 milligrams per kg weight of subject per administration. A typical dosage may be one 10-500 mg tablet taken once a day, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Dose levels can vary as a function of the specific compound, the severity of the symptoms, and the susceptibility of the subject to side effects. Some of the specific compounds that stimulate glutamatergic receptors are more potent than others. Preferred dosages for a given compound are readily determinable by those of skill in the art by a variety of means. One means is to measure the physiological potency of a given compound that is a candidate for administration. For example, excised patches and excitatory synaptic responses are measured in the presence of different concentrations of test compounds, and the differences in dosage response potency are recorded and compared. Potency can be evaluated in a variety of behavioral (exploratory activity, speed of performance) cognitive, and physical (excised patches and excitatory synaptic responses) tests.

### EXAMPLES

The following examples are offered to illustrate, but not to limit the claimed invention.

### Example 1

### Brain-Derived Neurotrophic Factor Restores Synaptic Plasticity in a Mouse Model of Huntington's Disease

Asymptomatic Huntington's Disease (HD) patients exhibit memory and cognition deficits that generally worsen with age. Related to this, long-term potentiation (LTP), a form of synaptic plasticity involved in memory encoding, is defective in HD mouse models well before motor deficits occur. Here we show that LTP is impaired in hippocampal slices from presymptomatic Hdh^{Q92} and Hdh^{Q111} knock-in mice and identify two contributing factors: 1) responses to theta burst stimulation (TBS) used to induce LTP are impaired in the mutants, and 2) TBS-induced actin polymerization in dendritic spines is greatly reduced. The decrease in actin polymerization and deficits in LTP stabilization were reversed by Brain-Derived Neurotrophic Factor (BDNF), concentrations of which were substantially reduced in Hdh^{Q111} mice. These results suggest that the HD mutation discretely disrupts processes needed to induce and consolidate LTP, with the latter effect likely arising from reduced BDNF expression. A potential therapeutic strategy is discussed.

Huntington's disease (HD) is caused by a mutation that expands the number of trinucleotide CAG repeats in the huntingtin protein gene (Vonsattel and DiFiglia (1998) J. Neuropathol. Exp. Neurol. 57: 369-384). Clinically, it is associated with severe motor disturbances and cognitive deficits that generally worsen with age (Id). The cognitive deficits include impairments to attention, executive function, visuospatial ability, semantic verbal fluency, and short and long-term memory. While some debate exists about when the cognitive problems first appear, several, especially those involving memory, can be discerned in asymptomatic gene carriers (Kirkwood et al. (2000) J. Neurol. Neurosurg. Psychiatry 69: 773-779; Lawrence et al. (1998) Brain Pathol. 121: 1329-1341; Lemiere et al. (2004) J. Neurol. 251: 935-942; Ho et al. 92003) Neurology 61: 1702-1706). The deficits are typically attributed to disturbances in the cortico-striatal system, but other structures involved in cognition, including amygdala and hippocampus, are affected in early stages of the disease (Rosas et al. (2003) Neurology 60: 1615-1620).

Impaired learning that occurs before motor symptoms or neuron loss has also been described for mouse models of HD (Lione et al. (1999) J. Neurosci. 19: 10428-10437; Van Raamsdonk et al. (2005) J. Neurosci. 25: 4169-4180; Mazarakis et al. (2005) J. Neurosci. 25: 3059-2066). These behavioral abnormalities are accompanied by the loss of long-term potentiation (LTP) (Usdin et al. (1999) Hum. Mol. Genet. 8: 839-846; Murphy et al. (2000) J. Neurosci. 20, 5115-5123), a form of synaptic plasticity widely regarded as a substrate for memory encoding, in the hippocampus, as well as by reductions in mossy fiber potentiation (Gibson et al. (2005) Eur. J. Neurosci. 22: 1701-1712). Reduced plasticity is evident weeks before the first signs of movement disorders, indicating that it is an early marker for HD rather than a secondary consequence of neurodegeneration. The reasons why LTP deteriorates in HD mouse models are unknown, but are likely to be important for understanding the cognitive problems that accompany the disease. Results from knock-in (72/80 CAG) mice point to a deficit in neurotransmitter mobilization (Usdin et al. (1999) Hum. Mol. Genet. 8: 839-846), but studies using transgenic (R6/2) mice suggest that processes that normally stabilize potentiation are impaired (Murphy et al. (2000) J. Neurosci. 20, 5115-5123). Importantly, LTP may be a particularly sensitive target for the early effects of the HD mutation because baseline physiological measures were normal in both mouse models.

Possibly related to the loss of plasticity is evidence that mutant huntingtin decreases expression of BDNF in the neocortex and hippocampus of humans (Ferrer et al. (2000) Brain Res. Brain Res. Rev. 866, 257-261; Zuccato et al. (2001) Science 293, 493-498) and mice (Zuccato et al. (2001) Science 293, 493-498; Gines et al. (2003) Hum. Mol. Genet. 12: 497-508; Zuccato et al. (2005) Pharmacol. Res. 52: 133-139). BDNF is an extremely potent, positive modulator of LTP when the potentiation effect is induced by naturalistic theta burst stimulation (TBS) (Bramham and Messaoud (2005) Prog. Neurobiol. 76: 99-125). The neurotrophin produces its effects, in part, by reducing after-hyperpolarizations that accompany theta burst responses (Kramar et al. (2004) J. Neurosci. 24: 5151-5161), and by facilitating the actin polymerization that occurs in dendritic spine heads immediately after stimulation (C.R. and G.L., unpublished observations). The first of these actions enhances the depolarization that induces LTP, while the second promotes an event essential to the stabilization (or consolidation) of the potentiated state (Bramham and Messaoud (2005) Prog. Neurobiol. 76: 99-125; Kramar et al. (2004) J. Neurosci. 24: 5151-5161; Lynch et al. (2007) Neuropharmacology, 52(1): 12-13; Kramar et al. (2006) Proc. Natl. Acad. Sci., USA, 103: 5579-5784). In the present studies, we tested if LTP induction, consolidation, or both are impaired in HD knock-in mice, and if BDNF restores normal plasticity.

### Results

Severe Impairment of LTP in Two Knock-in Mouse Models of HD

Hdh^{Q92} and Hdh^{Q111} mice, which have 92 or 111 CAG repeats inserted into the huntingtin (Hdh) gene under the influence of the endogenous promoter, were selected for these experiments because they closely resemble the genetic component of the human condition and, as with HD patients, have a delayed onset of overt symptoms (Menalled (2005) NeuroRx 2: 465-470). Mice were 8-weeks or 24-weeks old, time points that are almost a year in advance of the onset of motor disturbances.

Low frequency (baseline) stimulation was delivered to hippocampal slices from Hdh^{Q92}, Hdh^{Q111} and age-matched wild-type (WT) mice. Field excitatory post-synaptic potentials (fEPSPs) were comparable in HD and WT slices with regard to size (slope, amplitude) and waveform. Paired pulse facilitation (50 ms delays), a test for neurotransmitter mobilization, did not detectably differ between the groups (WT: 64±18%, n=6; combined HdhQ⁹², Hdh^{Q111}: 70±15%, n=9; means ±s.d.). Thus, baseline physiological measures were similar in WT and HD slices.

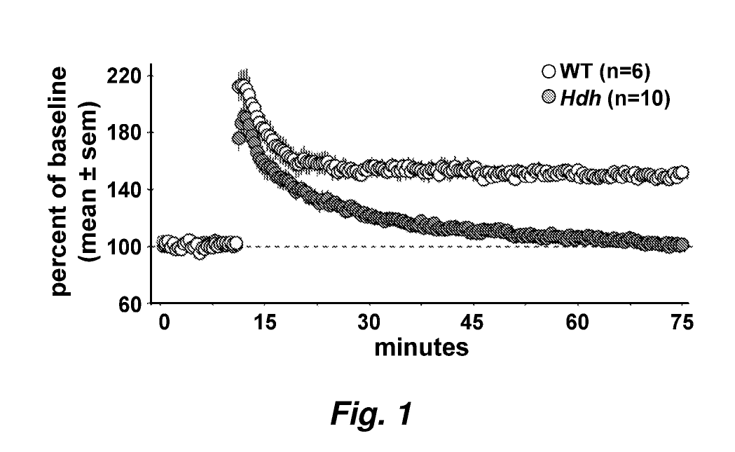

TBS was applied following a 20-30 min period of baseline recording; low frequency stimulation (3/min pulses) was resumed after TBS and responses were collected for an additional 60 min. In WT slices, TBS doubled the size of fEPSPs in the first minute after stimulation (FIG. 1), after which responses decayed to a stable level that was approximately 40% greater than the pre-TBS baseline. Slices from HD knock-in mice differed from controls in two ways: 1) the initial potentiation did not reach the levels seen in WT mice (P=0.019 at 20 sec post-TBS; two-tailed Student's t-test), and 2) the responses decayed rapidly (40 min) back to baseline; i.e., stable potentiation was not attained. The latter effect was highly significant (% LTP in WT versus HD mice at 45 min post-TBS: P<0.00001). These results indicate that expression of mutant huntingtin impairs the initial induction and, more severely, the stabilization of LTP.

LTP Induction and Consolidation Processes in Hdh^{Q111} Mice

Responses to TBS.

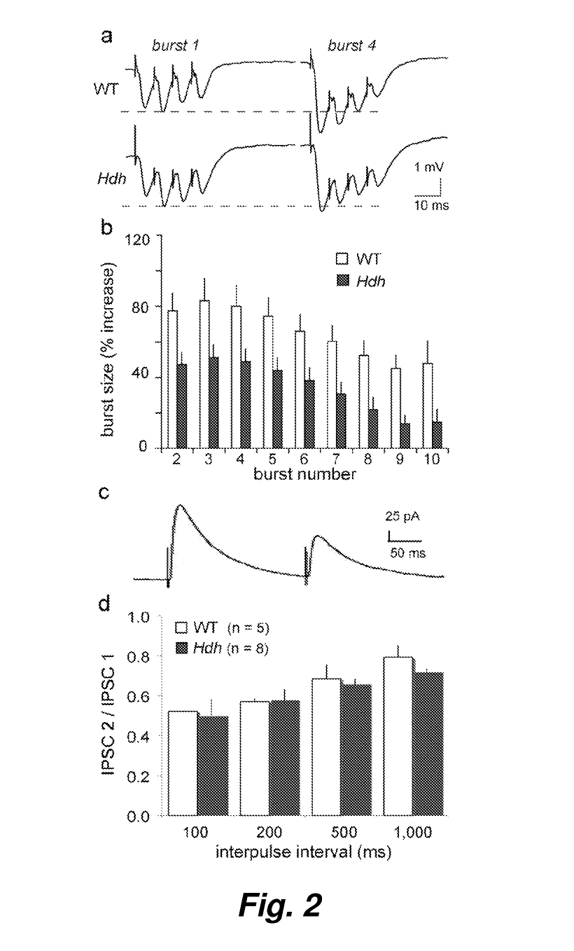

Events that occur during the two-second period of TBS can potently influence the threshold, initial expression, magnitude, and stability of LTP (Arai and Lynch (1992) Eur. J. Neurosci. 4: 411-419; Larson et al. (1986) Brain Res. 368: 347-350). In Hdh^{Q111} slices, the size (area) of the composite response to the first of ten stimulation bursts within a theta train did not differ from WTs (FIG. 2, panel a); group values were 60.9±7.8 mV ms for Hdh^{Q111} slices and 55.7±25.6 mV ms for WTs. This result suggests that calcium-dependent enhancement of neurotransmitter release, which normally occurs during TBS (Creager et al. (1980) J. Physiol. 299:409-424), and the inhibitory post-synaptic currents (IPSCs) that shape the first burst response (Larson et al. (1986) Brain Res. 368: 347-350; Mott and Lewis (1991) Science 252: 1718-1720), remain intact in Hdh^{Q111} mice. In marked contrast, the facilitation of the second and subsequent burst responses, each a composite of four closely spaced fEPSPs, was greatly attenuated in Hdh^{Q111} slices (FIG. 2, panel a, e.g. burst 4). The facilitation effect was quantified by expressing the areas of burst responses 2-10 as fractions of the area of the first burst response. As is evident (FIG. 2, panel b), within-train facilitation, described in numerous publications (Kramar et al. (2004) J. Neurosci. 24: 5151-5161; Rex et al. (2006) J. Neurophysiol. 96, 677-685), is substantially reduced in the Hdh^{Q111} relative to WT slices (mean facilitation across bursts 2-10 was 65.2±20.3% for WTs and 34.4±15.3% for Hdh^{Q111} mice; P=0.03).

Facilitation of responses during TBS arises because potent feedforward inhibitory post-synaptic currents (IPSCs), which are activated at the beginning of the first burst, enter a refractory period, and therefore exert a smaller current shunting effect on the second and subsequent burst responses (Larson et al. (1986) Brain Res. 368: 347-350; Mott and Lewis (1991) Science 252: 1718-1720). Thus, reduced within-train facilitation seen in Hdh^{Q111} mice could be due to a change in the refractoriness of IPSCs. We tested this by delivering two single stimulation pulses separated by 100-1,000 ms and measuring the size of the second feedforward IPSC relative to the first. The second IPSC in the Hdh^{Q111} slices was markedly reduced when initiated 100 ms after the first response (FIG. 2, panel c) as it was in WT slices. The depression of the second response was time dependent, with the greatest reduction occurring at 100 and 200 ms delays; the decrement at these intervals was 45±15% (n=8) in the Hdh^{Q111} slices and 45±3% (n=5) in WTs (FIG. 2, panel d). Factors other than IPSCs that might account for the loss of response facilitation during TBS are discussed below.

Actin Polymerization in Dendritic Spines.

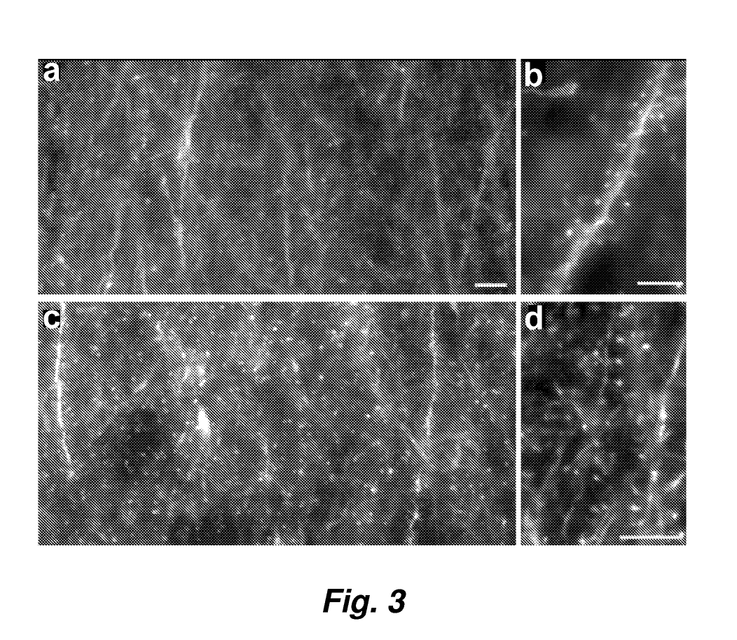

TBS causes actin to polymerize in adult spines of dendritic regions containing potentiated synapses (Lin et al. (2005) J. Neurosci. 25: 2062-2069) and this effect is closely related to the stabilization of LTP (Kramar et al. (2006) Proc. Natl. Acad. Sci., USA, 103: 5579-5784; Lin et al. (2005) J. Neurosci. 25: 2062-2069). The failure of LTP to stabilize in the HD knock-in mice prompted us to address whether actin polymerization is defective. First, we needed to establish whether our in situ method of applying rhodamine-conjugated phalloidin, a toxin that selectively binds to filamentous (F-) actin in its polymeric forms, effectively labels polymerized actin in dendritic spines of potentiated synapses in WT mice, as it does in rats (Kramar et al. (2006) Proc. Natl. Acad. Sci., USA, 103: 5579-5784). After low frequency stimulation or TBS, rhodamine-phalloidin was applied to hippocampal slices from WT mice. LTP induction with TBS was accompanied by a massive increase in the number of densely labeled puncta compared to slices given low frequency stimulation (FIG. 3, panels a and b). Similar increases were obtained using intracellular applications of rhodamine-phalloidin, demonstrating that, as with rat slices (Lin et al. (2005) J. Neurosci. 25: 2062-2069), the TBS-induced increase in F-actin labeling in mouse slices does not depend on the transport of the marker across cell membranes. The distribution of labeled puncta corresponded to that expected for potentiated synapses; i.e., high concentrations of labeling in the proximal portions of CA1 stratum radiatum with very few profiles in the more distal stratum molecular. The densely labeled puncta seen with intra- or extracellular applications of phalloidin after LTP induction had the size (about 1 μm in diameter), appearance (bulbous head, thin neck), and distribution (scattered along dendrites) expected for dendritic spines (FIG. 3, panels c and d).

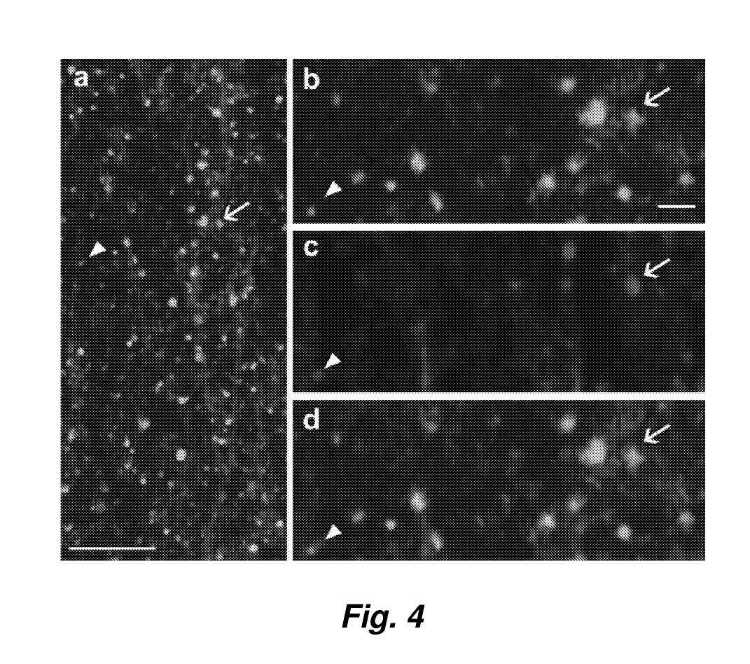

Further evidence that the phalloidin-labeled profiles are spines was obtained in double-labeling experiments using an antibody to PSD-95, a constituent of post-synaptic densities at glutamatergic synapses. Both phalloidin (red) and PSD-95 (green) labeling was abundant in the proximal portion of stratum radiatum in hippocampal field CA1 (FIG. 4, panel a; see FIG. 4, panels b and c for higher magnification images of PSD-95 or phalloidin labeling). The phalloidin-positive puncta typically overlapped with PSD-95 (FIG. 4, panel d, arrows) or ‘capped’ the scaffold protein attached to them (arrowhead).

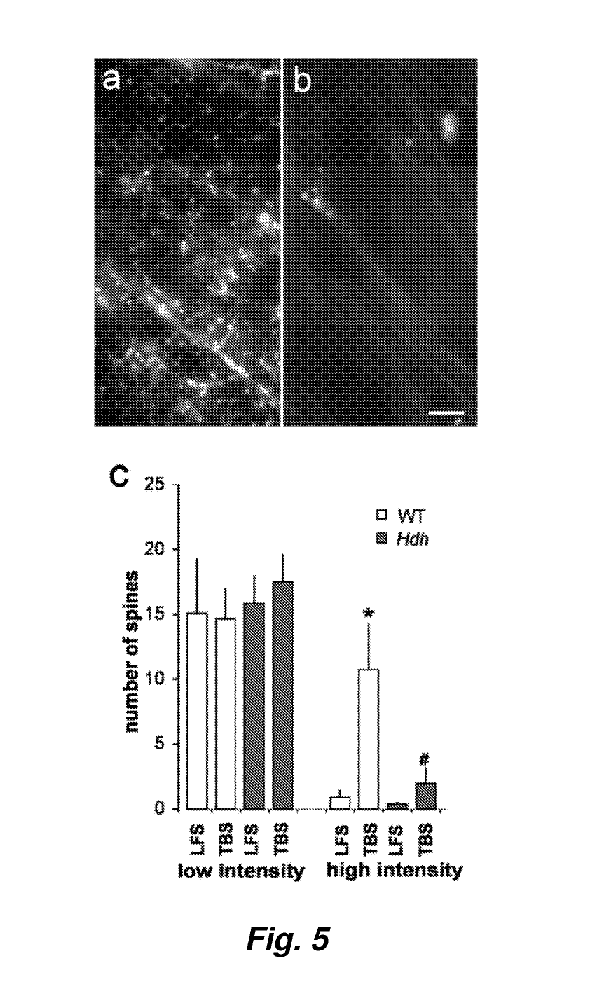

After establishing that TBS markedly increased polymerized actin in dendritic spines of WT mice, we tested for similar effects in Hdh^{Q111} slices. The increased number of densely labeled spines induced by TBS, as seen in WT slices (FIG. 5, panel a), was largely absent in Hdh^{Q111} mice (FIG. 5, panel b). Quantitative analyses were performed by categorizing the fluorescent labeling intensity of the spines into nine equal-sized bins ranging from very weak to very strong, and then counting the number of spines that fell into each bin. When the greatest numbers of labeled spines detected across the three lowest and three highest intensity bins were compared, it was evident that the incidence of weakly labeled spines was not influenced by genotype or by TBS (FIG. 5, panel c). That is, the number of such spines was about the same for WTs and Hdh^{Q111} mice, and this value was not affected by the induction of LTP. In contrast to effects of low frequency stimulation, TBS caused an approximately 10-fold increase in the incidence of densely labeled spines in WT slices (P=0.009, n=14) but did not have a statistically reliable effect in Hdh^{Q111} slices (P=0.37, n=13). The number of spines in the three highest intensity bins after TBS was significantly lower in Hdh^{Q111} compared to WT slices (P=0.02; Mann Whitney U-tests, two-tailed).

BDNF Restores LTP in Hippocampal Slices Prepared from Hdh^{Q111} Mice

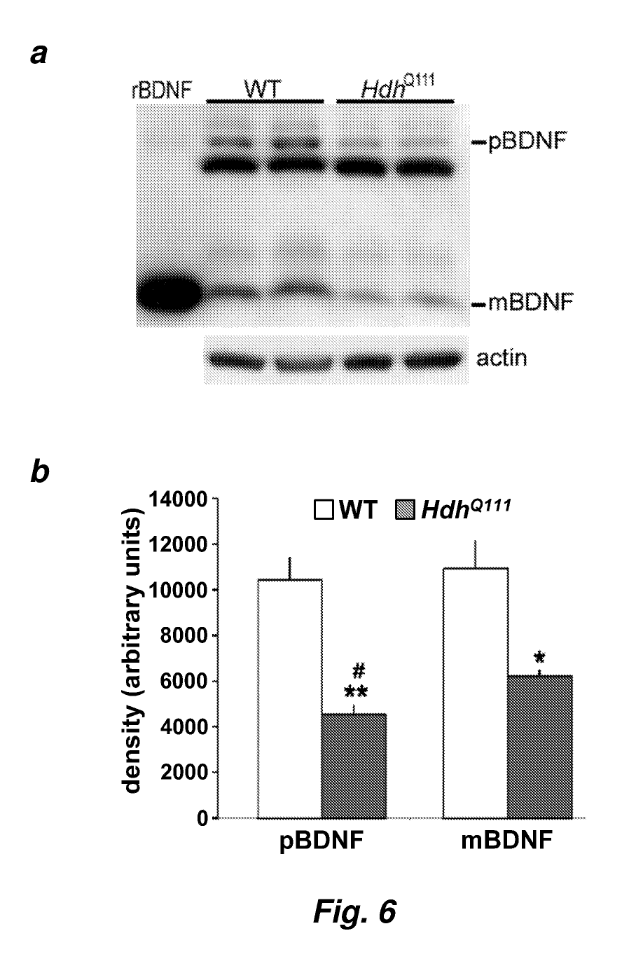

Previous studies showed that BDNF levels were substantially reduced in the neocortex and striatum of 20 week old Hdh^{Q111} mice (Gines et al. (2003) Hum. Mol. Genet. 12: 497-508). We tested if such effects are present in the hippocampus at 8 weeks, the age of Hdh^{Q111} mice used in the electrophysiological studies. Immunoblots of hippocampal samples from Hdh^{Q111} and WT mice showed that BDNF immunoreactivity was distributed across bands representing pro-BDNF and proteolytic fragments including a 14-kDa band, corresponding to mature BDNF protein (Mowla et al. (2001) J. Biol. Chem. 276: 12660-12666) (FIG. 6, panel a). Concentrations of both the pro- and mature BDNF were substantially reduced in Hdh^{Q111} mice. Densitometric analyses (FIG. 6, panel b) indicated that mature BDNF levels in hippocampus were 43±7% lower in Hdh^{Q111} (n=7) than in WT mice (n=8; P=0.002; one tailed t-test). The pro-BDNF immunoband, with an approximate mass of 29-kDa, was reduced by 57±11% in the mutants (P=0.0001). Differences between the groups were also significant when values were normalized to within-lane actin bands. Finally, the apparently greater loss of pro- vs. mature BDNF in Hdh^{Q111} mice, as seen in the group data (FIG. 6, panel b), proved to be reliable (P=0.007, paired Student's t-test).

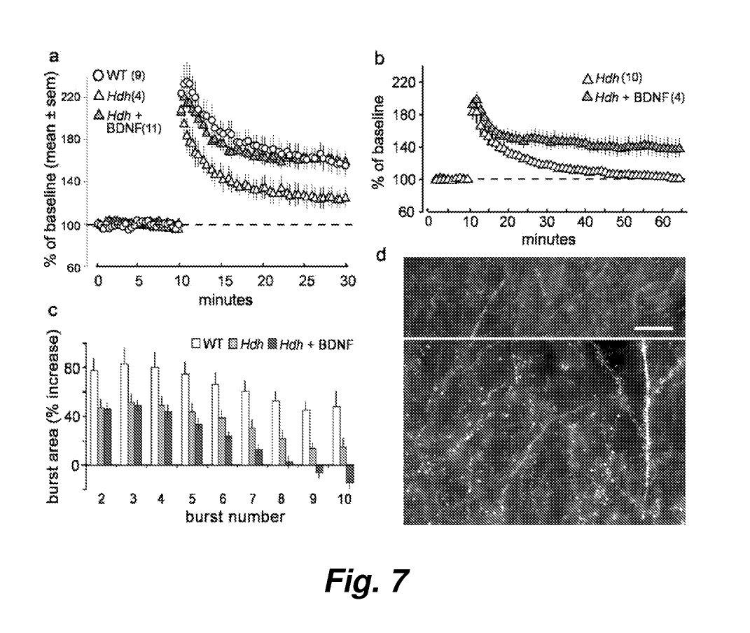

Since BDNF levels were substantially reduced in Hdh^{Q111} mice and the neurotrophin is a potent modulator of LTP (Kramar et al. (2006) Proc. Natl. Acad. Sci., USA, 103: 5579-5784), we tested if BDNF (2 nM) would reverse LTP deficits in HD mice. Infusing BDNF for 60 min did not detectably change baseline transmission in Hdh^{Q111} or WT slices but, had potent effects on LTP in the mutants (FIG. 7, panel a). Slices from Hdh^{Q111} mice that were not treated with BDNF (n=4) exhibited decremental potentiation (FIG. 7, panel a), as described above, and were clearly different from WT slices (n=9) by 20 min post-TBS (Hdh^{Q111}: 24.3±17.1% versus WT: 56.5±30.5%). In contrast, LTP in BDNF-treated Hdh^{Q111} slices (59.9±28.3% at 20 min post-TBS; n=11) was equivalent to WTs and significantly greater than that seen in the untreated HD group (P=0.016). Post-TBS responses were followed for 60 min in a subgroup of the BDNF-treated Hdh^{Q111} slices (n=4; FIG. 7, panel b) and stable potentiation was present at the end of recording. The percent potentiation of BDNF-treated slices 45 min after TBS was much greater than that recorded for untreated Hdh^{Q111} slices (43.6±23.5% and 6.0±12.1%, respectively; P<0.002).

BDNF could restore LTP in Hdh^{Q111} slices by enhancing within-train facilitation of theta burst responses, as has been described for rats (Kramar et al. (2004) J. Neurosci. 24: 5151-5161). This idea is particularly relevant given that within-train facilitation is impaired in Hdh^{Q111} mice (see FIG. 2, panel b). However, BDNF had no effect on the genotype-specific loss of facilitation from the first to subsequent burst responses (FIG. 7, panel c). Facilitation of the second burst response was 77.3±20.5% for WTs, 46.9±16.8% for untreated Hdh^{Q111} slices, and 46.1±10.2% for BDNF-treated Hdh^{Q111} slices (P<0.03 for BDNF-treated Hdh^{Q111} compared to WT slices). The loss of facilitation was even more pronounced for the later bursts in the stimulation train; e.g., burst 9 was 45.0±15.4% greater than burst 1 in WT slices and −5.8±9.3% for BDNF-treated Hdh^{Q111} slices (P<0.001). In all, BDNF did not facilitate responses to any stimulation burst in the theta train in Hdh^{Q111} slices.

The failure of BDNF to restore within-train burst facilitation in Hdh^{Q111} mice suggests that it exerts its positive effect on LTP by reversing the deficits in actin polymerization. To test this, rhodamine-phalloidin was applied after physiological recordings were collected from WT and Hdh^{Q111} slices with and without BDNF treatment. Low frequency stimulation did not elicit changes in phalloidin labeling of BDNF-treated or untreated slices from either genotype. As described above, TBS induced robust LTP in slices from Hdh^{Q111} mice treated with BDNF but not in untreated slices. LTP restoration was accompanied by a marked increase in densely labeled spines in BDNF-treated (FIG. 7, panel d, bottom), compared to untreated slices (FIG. 7, panel d, top). The number of phalloidin-labeled spines in the high-intensity bins was 40.5±39.3 per 550 μm² in Hdh^{Q111} slices treated with BDNF (n=13) and 2.0±4.6 for those without BDNF (n=13; P<0.003, Mann Whitney U-test). Thus, the restorative effect of BDNF on LTP stabilization in Hdh^{Q111} mice may be due its effects on processes mediating actin polymerization.

### DISCUSSION

Cognitive deficits are present in HD gene carriers and early stage patients well before the onset of the motor symptoms that define the disease (Kirkwood et al. (2000) J. Neurol. Neurosurg. Psychiatry 69: 773-779; Lawrence et al. (1998) Brain Pathol. 121: 1329-1341; Lemiere et al. (2004) J. Neurol. 251: 935-942; Ho et al. 92003) Neurology 61: 1702-1706). A recent longitudinal study concluded that the problems are progressive, even over periods as short as three years, and that memory losses are the earliest cognitive manifestations of HD (Lemiere et al. (2004) J. Neurol. 251: 935-942). These findings suggest that HD begins with a discrete disturbance of plasticity and then progresses to motor pathology and neurodegeneration.

Prior studies indicated that LTP, a form of synaptic plasticity widely regarded as the substrate for memory encoding, is severely impaired in hippocampal field CA1 in HD mouse models (Usdin et al. (1999) Hum. Mol. Genet. 8: 839-846; Murphy et al. (2000) J. Neurosci. 20, 5115-5123). Paired-pulse facilitation was depressed along with LTP in HD knock-in (72/80 CAG) mice suggesting that the HD mutation alters release kinetics (Usdin et al. (1999) Hum. Mol. Genet. 8: 839-846). Although the point was not tested, a presynaptic deficit would presumably disturb frequency facilitation of post-synaptic responses, and thus reduce the depolarization needed to trigger LTP. A separate study found that R6/2 and WT mice are comparable in their basic synaptic physiology, including presynaptic neurotransmitter mobilization and release (Murphy et al. (2000) J. Neurosci. 20, 5115-5123). This suggests a post-synaptic locus for the LTP deficit. NMDA-receptor mediated currents also appeared normal in R6/2 mice, again indicating that LTP processes downstream of induction (i.e., expression or stabilization) are impaired. In all, previous studies indicate that LTP deficits are present in HD mouse models but disagree as to whether they reflect a pre- or post-synaptic problem.

The present experiments used the naturalistic TBS pattern to induce LTP in hippocampal slices prepared from HD knock-in mice. Most of the experiments were performed with 8-week old Hdh^{Q111} mice, so as to test for deficits that are evident before overt motor disturbances and during the transition from late development to early adulthood. These conditions allow the reasonable assumption that the results are relevant to the early appearance of memory problems in HD patients. LTP was severely impaired in HD mice without evidence of presynaptic disturbances or changes in the waveform of the post-synaptic responses. Moreover, the size and shape of the composite response to a single burst of afferent stimulation were normal, as were feedforward inhibitory potentials. However, the facilitation of burst responses that normally occurs during a theta train was markedly reduced in the HD knock-in mice. Burst facilitation causes greater depolarization which enhances the opening of NMDA receptors triggering LTP (Larson and Lynch (1988) Brain Res. 441: 111-118). Thus, impaired burst responses in Hdh^{Q111} mice probably contribute to the defective LTP.

One explanation for the modified burst responses is that IPSCs, which accompany individual burst responses (Larson et al. (1986) Brain Res. 368: 347-350), are altered. IPSCs are reduced during TBS because inhibitory synapses become refractory after they are activated during the first theta burst response, an effect caused by stimulation of presynaptic autoreceptors (Mott and Lewis (1991) Science 252: 1718-1720). Thus, deficits in the processes controlling the strength and duration of this refractory effect could impair burst facilitation. Tests of this idea, however, proved negative: IPSCs were as refractory in Hdh^{Q111} as in WT mice. The remaining factor controlling within-train changes in burst response characteristics is the complex sequence of after-hyperpolarizations triggered by the first burst response (Arai and Lynch (1992) Eur. J. Neurosci. 4: 411-419; Sah and Bekkers (1996) J. Neurosci. 16: 4537-4542). In any event, the pronounced impairment in LTP found in Hdh^{Q111} mice is associated with surprisingly discrete post-synaptic defects.

Possibly related to disturbances in theta burst responses, BDNF levels were reduced in hippocampus of 8 week old Hdh^{Q111} mice. Earlier reports found low levels of the neurotrophin in neocortex and striatum of somewhat older knock-in mice (Gines et al. (2003) Hum. Mol. Genet. 12: 497-508). BDNF enhances burst response facilitation during TBS by suppressing the above discussed after-hyperpolarizations (Kramar et al. (2004) J. Neurosci. 24: 5151-5161) and, as expected from this, promotes the induction of LTP (for a review see Bramham and Messaoud (2005) Prog. Neurobiol. 76: 99-125). Given that the neurotrophin is released by TBS (Aicardi et al. (2004) Proc. Natl. Acad. Sci., USA, 101: 15788-15792; Balkowiec and Katz (2000) J. Neurosci. 20: 7417-74123), these past observations suggest a unifying explanation for the pattern of results obtained in Hdh^{Q111} mice; i.e., reduced BDNF production removes a factor that positively modulates theta burst facilitation and thus the induction of LTP. If so, then infusing BDNF should rescue LTP in the HD mice by restoring normal responses to TBS. The first of these predictions was confirmed: Hdh^{Q111} slices exposed to BDNF, at a physiologically plausible concentration (2 nM), exhibited robust and stable LTP following TBS. Unexpectedly, however, the rescue of potentiation was not accompanied by the return of normal within-train facilitation. The latter finding strongly suggests that signaling from BDNF's trkB receptor to the potassium channels that mediate after-hyperpolarizations is in some way disturbed by mutant huntingtin. It also indicates that BDNF's influence on events following burst responses is likely to be responsible for the rescue of plasticity in Hdh^{Q111} slices.

The above conclusion led us to investigate whether BDNF affects the second of the two LTP-related processes that were defective in Hdh^{Q111} slices, namely TBS-induced actin polymerization. Tests of this idea were positive: TBS produced a pronounced increase in the number of spines with dense concentrations of F-actin in Hdh^{Q111} slices that had been pretreated with BDNF compared to untreated slices. Multiple lines of evidence indicate that actin polymerization is an essential step in the stabilization (consolidation) of LTP (Kramar et al. (2006) Proc. Natl. Acad. Sci., USA, 103: 5579-5784; Ackermann and Matus (2003) Nat. Neurosci. 6: 1194-200; Fukazawa et al. (2003) Neuron 38: 447-460; Okamoto et al. (2004) Nat. Neurosci. 7: 1104-1112). For example, agents that disrupt actin polymerization block LTP consolidation (Kramar et al. (2006) Proc. Natl. Acad. Sci., USA, 103: 5579-5784; Fukazawa et al. (2003) Neuron 38: 447-460) while treatments that disrupt consolidation eliminate TBS-induced actin polymerization (Kramar et al. (2006) Proc. Natl. Acad. Sci., USA, 103: 5579-5784). Moreover, actin polymerization has the same threshold (number of theta bursts) for induction as does LTP and becomes resistant to disruption over the same time period that the potentiation effect consolidates (Id.). The absence of TBS-induced actin polymerization can thus explain why the sizeable initial potentiation found in Hdh^{Q111} slices decays back to baseline rather than stabilizing, while recovery of polymerization accounts for the rescue of LTP by BDNF.

The deficits in actin polymerization found in Hdh^{Q111} mice also provide a possible explanation for the abnormal spine morphology found in striatal and cortical neurons of HD transgenic mouse models (Guidetti et al. (2001) Exp. Neurol. 169: 340-350; Klapstein et al. (2001) J. Neurophysiol. 86, 2667-2677; Spires et al. (2004) Eur. J. Neurosci. 19: 2799-807) as well as in HD patients (Ferrante et al. (1991) J. Neurosci. 11: 3877-3887; Graveland et al. (1985) Science 227: 770-773). The actin cytoskeleton regulates the shape of dendritic spines (Fischer et al. (2000) Nat. Neurosci. 3: 887-894; Star et al. (2002) Nat. Neurosci. 5: 239-246), at least in developing tissue, and deficiencies in activity-dependent filament assembly would be expected to eventually cause aberrant morphology. It seems unlikely that spines in hippocampus were grossly abnormal in the 8 week old Hdh^{Q111} mice used in the present studies because baseline synaptic potentials were not detectably different from those in WT mice. Future studies are needed to address the possibility that deficits in TBS-induced actin polymerization and LTP emerge during the juvenile period and are followed in adulthood by significant disturbances to spine morphology.

The restorative effect of BDNF on LTP in Hdh^{Q111} mice raises the question of whether HD-related cognitive impairments can be overcome by up-regulating production of the neurotrophin. Various methods for elevating BDNF levels have been reported (e.g. antidepressant drugs, seizures) but most of these have unacceptable side-effects, especially for long-term applications. Exercise (Cotman and Berchtold (2002) Trends Neurosci. 25: 295-301; Pang et al. (2006) Neuroscience, 141(2): 569-584) and enriched environments (Spires et al. (2004) J. Neurosci. 24: 2270-2276) also increase BDNF production, but these effects occur in comparison to sedentary or non-enriched controls, and HD patients are not inactive/sensory deprived prior to the emergence of motor disturbances. Alternatively, work from several groups (Lauterbom et al. (2000) J. Neurosci. 20: 8-21; Legutko et al. (2001) Neuropharmacology 40: 1019-1027; O'Neill et al. (2005) CNS Drug Rev. 11: 77-96) has shown that BDNF mRNA and protein concentrations can be substantially increased in hippocampus (both in vitro and in vivo) by ampakines, a large family of compounds that positively modulate AMPA-type glutamate receptors and the fast EPSPs they mediate. Moreover, chronic use of ampakines does not produce significant side-effects in rats, monkeys, or humans (Lynch (2996) Curr. Opin. Pharmacol. 6: 82-88). Accordingly, ampakines appear to provide a viable therapeutic approach.

In summary, we found that mutant huntingtin negatively affects, quite likely via separate pathways, key steps in the sequences responsible for inducing and consolidating LTP. The induction problem appears to be confined to an event that emerges after the first theta burst response and, as judged from its resistance to BDNF, may involve kinase signaling cascades. Perhaps the most parsimonious explanation for the defect in LTP consolidation is that the amount of BDNF released by TBS in HD knock-in mice is too low to activate neurotrophin sensitive pathways that promote actin filament assembly and thereby contribute importantly to the production of stable LTP (Zuccato et al. (2003) Nat. Genet. 35: 76-83). Combined, the two effects of the HD mutation result in a severe impairment to synaptic plasticity.

### Methods

Mice and Genotyping.

Hdh^{Q92} and Hdh^{Q111} mice have 92 and 111 CAG repeats, respectively, inserted into the huntingtin gene under the influence of the endogenous promoter (for a review see 2). Homozygous Hdh^{Q92} and Hdh^{Q111} breeding pairs were purchased from Jackson Laboratories and maintained as an inbred colony. WT mice from the same background strain (C57BL/6J) and vendor were used as controls. Periodic genotyping used standard polymerase chain reaction procedures and the following primers: 5′-GGC TGA GGA AGC TGA GGA G-3′ (SEQ ID NO:1), 5′-GTC CTG ACA TCG GGA AAG AG-3′, and 5′-GTT CCT CTG CCG GAC CTG-3′ (SEQ ID NO:2).

Physiology.

Acute hippocampal slices were prepared, as described elsewhere (Kramar et al. (2004) J. Neurosci. 24: 5151-5161), from 8 week old Hdh^{Q111}, 24 week old Hdh^{Q92} and age-matched WT mice and maintained at 32° C. in an interface chamber of local design. Synaptic responses (fEPSPs) were generated by stimulating the Schaffer-commissural afferents to the apical dendrites of field CA1b pyramidal cells using stimulating electrodes positioned in fields CA1a and CA1c. For baseline recording, fEPSPs were set to 30% of the maximum responses and low frequency stimulation (3 pulses/min) was delivered. The slope of the descending phase of the fEPSP was used as a measure of response size with all values normalized to a 15 min baseline period collected 1-2 h after slice preparation. LTP was induced with a single TBS train (ten bursts of 4 pulses at 100 Hz, inter-burst interval of 200 ms). BDNF (2 nM) was prepared and delivered to slices as described previously (Menalled (2005) NeuroRx 2: 465-470).

For whole cell recordings, CA1 pyramidal neurons were visualized with an infrared microscope in DIC configuration and recordings made with 3-5 Mohm pipettes. Holding potentials were set to −70 mV after correcting for the junction potential. Currents were sampled with a patch amplifier with a 4-pole low-pass Bessel filter at 2 kHz and digitized at 10 kHz.

For all electrophysiology experiments, results are summarized in the text as a percent increase in responses from baseline. Data are presented as mean ±s.d. in the text and mean ±s.e.m. in the figures.

In Situ Labeling of F-Actin.

Physiological recording and delivery of TBS or low frequency stimulation was performed as described above. Starting 20 min after TBS, rhodamine-phalloidin (6 μM/2-4 μl; from Sigma, St. Louis, Mo., or Invitrogen, Carlsbad, Calif.) was applied topically (Kramar et al. (2006) Proc. Natl. Acad. Sci., USA, 103: 5579-5784) via micropipette 3 times separated by 3 min. To test the effects of BDNF on actin polymerization, BDNF (2 nM) or artificial cerebral spinal fluid was delivered to WT and Hdh^{Q111} slices via a reperfusion pump system (Cole-Palmer, Vernon Hills, Ill.) at 1 ml/min 1-2 h before recording. Slices were then fixed in 4% paraformaldehyde, cryoprotected with 20% sucrose, sectioned at 20 μm on a freezing microtome, and coverslipped with Vectashield (Vector Laboratories, Burlingame, Calif.).

Sections were examined with epifluorescence illumination using an Olympus AX70 photomicroscope. Quantitative analyses were carried out on three serial sections situated 20 to 80 μm below the surface of original slice. A series of 15-20 high resolution digital photomicrographs were taken at 1 μm focal (Z-axis) plane steps through each section (Z-stacks). Camera exposure time was adjusted for each experiment so that approximately 4-8 spines could be visualized in the sample field of control slices. Subsequent images intended for comparison were then collected with the same illumination and exposure settings. The Z-stacks were collapsed into a single image by extended focal imaging using Microsuite FIVE (Soft Imaging Systems, Lakewood, Colo.). These images were then converted to grayscale and intensity levels were cropped at values determined for each experiment to visualize low-intensity labeling.

Labeled spine-like structures were measured and counted from a 550 μm² sampling zone in the proximal stratum radiatum between the two stimulating electrodes using in-house software described in detail previously (Kramar et al. (2006) Proc. Natl. Acad. Sci., USA, 103: 5579-5784; Lin et al. (2005) J. Neurosci. 25: 2062-2069). Counting was done blindly on batches of slices that had been sectioned and stained together. Intensity thresholds based on a pixel intensity unit scale (8-bit) were applied to identify spine-like structures at varying levels of label intensity. Digital images of objects included in the counts were overlaid semi-transparently with the original photomicrographs to confirm that they were spines. Spine counts from each of the three series of sections were averaged to produce a representative value for each slice.

For double-labeling experiments, slices were labeled with rhodamine-phalloidin (12 μM) and prepared for histology, as described above. Sections were then incubated (1 h, room temperature) with the rabbit polyclonal anti-PSD-95 (MAB1598; Chemicon, Temecula, Calif.) at 1:100 in 0.1 M phosphate buffer (PB) containing 4% bovine serum albumin and 0.3% Triton X-100 (PBT). Slides were rinsed in 0.1 M PB, incubated (45 min, room temperature) in fluorescein anti-rabbit IgG (1:200; Vector) in PBT and rinsed again. Laser scanning confocal microscopy was used to assess double labeling. Tissue sections were qualitatively analyzed from image Z-stacks collected at 60× magnification. Image processing was performed with Photoshop 6.0 (Adobe Systems). Figures show single 1 μm thick optical sections.

Western Blots.

Endogenous BDNF levels were assessed using western blots with a BDNF antibody (1:1,000; Santa Cruz, Calif.) that recognizes both mature and pro-forms of the protein. Hippocampus of 8 week old Hdh^{Q111} (n=7) and WT (n=8) mice were homogenized in NP40 cell lysis buffer (Biosource, Camarillo, Calif.) with protease inhibitor cocktail (Sigma, Cat #P2714) and 1 mM phenylmethanesulfonyl fluoride (Sigma). Protein levels were measured using the BioRad Protein Assay (BioRad Laboratories, Hercules, Calif.). Protein samples (25 μg/lane) were then separated by 15% PAGE, transferred to nitrocellulose membranes (BioRad) and immunoreactive bands were visualized using the enhanced chemiluminescence ECL Detection System (Amersham Biosciences, Buckinghamshire, UK). As a positive control, recombinant human BDNF (Chemicon) was loaded on the same gels as samples. To control for loading variations, blots were stripped and reprobed with anti-actin (1:2,000; Sigma).

Densitometric analyses of immunoreactive bands were performed using NIH Image software. For each blot, the densities of the BDNF immunoreactive bands were expressed as a fraction of the actin immunoreactive band in the same lane; samples were run 3 separate times and results averaged. Statistical analyses were run on both raw and actin-normalized values for pro- and mature BDNF bands. Significance of the effect of genotype was evaluated using a one-tailed Student's t-test.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of preserving or improving cognitive function in a presymtomatic or asymptomatic mammal having one or more mutations predisposing said mammal to Huntington's disease, said method comprising: maintaining or increasing the BDNF level or activity in the brain of said mammal.

2. The method of claim 1, wherein said mammal shows no substantial neural degeneration.

3. The method of claim 1, wherein said mammal shows essentially no measurable neural degeneration.

4. The method of claim 1, wherein said mammal is a mammal diagnosed as having one or more mutations in the huntingtin gene.

5. The method of claim 1, wherein said mammal is a mammal diagnosed as having one or more mutations in the huntingtin gene prior to maintaining or increasing said BDNF level or activity.

6. The method of claim 1, wherein said mammal is a mammal not having a diagnosis of depression.

7. The method of claim 1, wherein said mammal is a mammal not having a diagnosis of depression or other psychiatric disorder.

8. The method of claim 1, wherein said mammal is a mammal diagnosed as having one or more mutations in the huntingtin gene prior to maintaining or increasing said BDNF level or activity

9. The method of claim 4, wherein said mutation is a trinucleotide repeat expansion in the huntingtin gene.

10. The method of claim 1, wherein said maintaining or increasing the BDNF level or activity comprises administering a glutamate AMPA receptor modulators (ampakines) to said mammal in an amount sufficient to upregulate expression or activity of BDNF in said mammal.

11. The method of claim 1, wherein said maintaining or increasing the BDNF level or activity in said mammal comprises restricting diet and/or increasing physical exercise of said mammal.

12. The method of claim 1, wherein said maintaining or increasing the BDNF level or activity in said mammal comprises administering to said mammal one or more agents selected from the group consisting of an anti-depressant drug or an anti-anxiolytic drug, an anti-psychotic drug, an acetylcholinesterase inhibitor.

13. The method of claim 12, wherein the agent comprises fluoxetine, desipramine, or 2-methyl-6-(phenylethynyl)-pyridine).

14. The method of claim 12, wherein the agent comprises afobazole.

15. The method of claim 12, wherein the agent comprises a histone deacetylase inhibitors (e.g. sodium butyrate).

16. The method of claim 12, wherein the agent comprises a neuropeptide whose expression is regulated by cocaine- or other amphetamine.

17. The method of claim 12, wherein the agent comprises cystamine or nicotine.

18. The method of claim 12, wherein the agent comprises quetiapene or venlafaxine).

19. The method of claim 12, wherein the agent comprises huperzine A

20. The method of claim 12, wherein the agent comprises a monocyclic or bicyclic loop mimetic of BDNF.

21. The method of claim 12, wherein the agent comprises estrogen or adrenocorticotropin.

22. The method of claim 12, wherein the agent comprises dopamine, norepinephrine, LDOPA, serotonin, or analogues thereof.

23. The method of claim 12, wherein the gent comprises Semax.

24. The method of claim 12, wherein the agent comprises a compound that increases the activity of BDNF through up-regulating the BDNF receptor.

25. The use of a compound that increases the level or activity of BDNF in a mammal in the manufacture of a medicament for the treatment or prevention of cognitive dysfunction in a pre- or asymptomatic mammal having one or more mutations in the Huntington gene.

26. The use of claim 25, wherein said compound is an ampakine.

27. A kit for the treatment or prevention of cognitive dysfunction in a pre- or asymptomatic mammal having one or more mutations in the Huntington gene, said kit comprising:
a container containing one or more agents that increase the expression or activity of BDNF in a mammal; and
instructional materials teaching the use of said agents to mitigate or prevent cognitive disorder in a presymptomatic or asymptomatic mammal diagnosed with one or more mutations in a Huntington gene.


SEQUENCE LISTING (3 sequences)

SEQ ID NO: 1 - DNA, 19 bp, Artificial
  Synthetic oligonucleotide PCR Primer
ggctgaggaagctgaggag

SEQ ID NO: 2 - DNA, 17 bp, Artificial
  Synthetic oligonucleotide PCR Primer
ctgacatcgggaaagag

SEQ ID NO: 3 - DNA, 18 bp, Artificial
  Synthetic oligonucleotide PCR Primer
gttcctctgccggacctg